(19) United States
(12) Patent Application Publication
(10) Pub. No.: US 2013/0195764 A1
(43) Pub. Date: Aug. 1, 2013

(54) Nanoparticle Targeting to Ischemia for Imaging and Therapy

(71) Applicants: Jaeyun Kim, Gyeonggi-do (KR); Lan Cao, Stoughton, MA (US); David J. Mooney, Sudbury, MA (US)

(72) Inventors: Jaeyun Kim, Gyeonggi-do (KR); Lan Cao, Stoughton, MA (US); David J. Mooney, Sudbury, MA (US)

(73) Assignee: PRESIDENT AND FELLOWS OF HARVARD COLLEGE, Cambridge, MA (US)

(21) Appl. No.: 13/641,971

(22) PCT Filed: Apr. 20, 2011
(86) PCT No.: PCT/US11/33272

§ 371 Date: Apr. 17, 2013

Related U.S. Application Data
(60) Provisional application No. 61/342,885, filed on Apr. 21, 2010.

(51) Int. Cl. A61K 49/00; A61K 38/18

(52) U.S. Cl. CPC A61K 49/005; A61K 38/1866
USPC 424/9.6; 530/399; 424/172.1; 514/8.1

(57) ABSTRACT

The invention provides compositions and methods that provide a solution to the difficulties in diagnosing ischemia, e.g., identifying specific affected anatomical areas, and treating ischemic tissue so as to minimize damage and promote healing of damaged tissue in a subject such as a human or other animal.

### RELATED APPLICATIONS

This application claims priority to U.S. Ser. No. 61/342,885, filed on Apr. 21, 2010, which is incorporated herein by reference in its entirety.

### STATEMENT AS TO FEDERALLY SPONSORED RESEARCH

This invention was made with U.S. Government support under Grant Number R01 DE0 13349 from the National Institutes of Health. The Government has certain rights in the invention.

### FIELD OF THE INVENTION

The invention relates to diagnostic and therapeutic methods of targeting ischemic tissue.

### BACKGROUND

Ischemia is a condition in which the blood flow (and thus oxygen) is restricted to a part of the body. Cardiac ischemia is characterized by a lack of blood flow and oxygen to the heart muscle. When arteries of the heart are narrowed, less blood and oxygen reaches the heart muscle, which leads to coronary artery disease and coronary heart disease. This condition can ultimately lead to heart attack. However, ischemia is a feature of not only heart diseases, but transient ischemic attacks, cerebrovascular accidents, e.g., stroke, ruptured arteriovenous malformations, and peripheral artery occlusive disease. Although the heart, the kidneys, and the brain are among the organs that are the most sensitive to inadequate blood supply, atherosclerosis of the extremities also causes ischemia in peripheral arterial disease (PAD)/peripheral vascular disease (PVD). Mild PAD may be asymptomatic or cause intermittent pain, whereas more serious PAD may cause rest pain in legs and toes, skin atrophy, hair loss, cyanosis, ischemic ulcers, and gangrene. As such, there is a pressing need in the art for new strategies to diagnose and treat ischemic tissues.

### SUMMARY OF THE INVENTION

The invention provides compositions and methods that provide a solution to the difficulties in diagnosing ischemia, e.g., identifying specific affected anatomical areas, and treating ischemic tissue so as to minimize damage and promote healing of damaged tissue in a subject. The subject is preferably a mammal in need of such treatment, e.g., a subject that has been diagnosed with an acute or chronic ischemic condition or a predisposition thereto. The mammal can be, e.g., any mammal, e.g., a human, a primate, a mouse, a rat, a dog, a cat, a horse, as well as livestock or animals grown for food consumption, e.g., cattle, sheep, pigs, chickens, and goats. In a preferred embodiment, the mammal is a human.

Accordingly, the invention features a composition, e.g., a pharmaceutical composition, comprising a nanoparticle with an angiogenesis-promoting factor linked thereto. Such molecules (and their amino acid (aa) and nucleic acid (na) sequences) are well known in the art. For example, the angiogenesis-promoting factor comprises a growth factor or cytokine. Exemplary factors include vascular endothelial growth factor (e.g., VEGFA; GenBank Accession Number: (aa) AAA35789.1 (GI:181971), (na) NM_{—}001171630.1 (GI:284172472), incorporated herein by reference), basic fibroblast growth factor (bFGF; GenBank Accession Number: (aa) AAB21432.2 (GI:8250666), (na) A32848.1 (GI:23957592), incorporated herein by reference), platelet derived growth factor (PDGF; GenBank Accession Number: (aa) AAA60552.1 (GI:338209), (na) NM_{—}033023.4 (GI:197333759), incorporated herein by reference), placental growth factor (PLGF; GenBank Accession Number: (aa) AAH07789.1 (GI:14043631), (na) NM_{—}002632.4 (GI:56676307), incorporated herein by reference), Angiopoietin (e.g., Ang-1; GenBank Accession Number: (aa) AAI52420.1 (GI:156230950), (na) NM_{—}001146.3 (GI:21328452), incorporated herein by reference), stromal-derived factor (e.g., SDF-2; GenBank Accession Number: (aa) AAP35355.1 (GI:30582257), (na) NM_{—}006923.2 (GI:14141194), incorporated herein by reference), granulocyte-macrophage colony stimulating factor (GM-CSF; GenBank Accession Number: (aa) AAA52578.1 (GI:183364), (na) M11220.1 (GI:183363), incorporated herein by reference, and granulocyte colony stimulating factor (G-CSF; GenBank Accession Number: (aa) CAA27290.1 (GI:732764), (na) X03438.1 (GI:31689), incorporated herein by reference).

Preferably, VEGF is linked to the nanoparticle. The linkage is covalent or non-covalent, with covalent bonds being preferred. For example, the VEGF is linked to the nanoparticle via a thio, e.g., S—S, bond. The nanoparticles are optionally customized to include moieties, such as an antibody or antigen-binding fragment thereof, that bind to proteins that are expressed or secreted at ischemic sites. For example, the nanoparticle further comprises a composition that binds to or associates with ICAM-1, P-selectin, E-selectin, or αᵥβ₃ integrin. Preferably, the nanoparticle is non-liposomal in nature. For example, the composition does not comprise small unilamellar vesicles containing phospholipids and aliphatic side chains.

A method for preferentially promoting angiogenesis at an ischemic anatomical site compared to a non-ischemic site is carried out by administering to a subject identified as suffering from or suspected of suffering from ischemia the nanoparticle described above. The particles are administered systemically, regionally, or locally (e.g., directly to the site of ischemic tissue. Such nanoparticles preferentially localize to an ischemic anatomical site compared to a non-ischemic site. In a variation of this method, a target site that is not characterized as ischemic or is not characterized as severely ischemic is pre-treated with an agent to induce a physiological environment to which the nanoparticles localize. Thus, a method for promoting angiogenesis at a target anatomical site in a subject is carried out by locally administering to the target site an enhanced permeability and retention (EPR)-inducing agent and subsequently administering to the subject the therapeutic nanoparticle composition described above, e.g., VEGF-conjugated nanoparticle.

Nanoparticles to which a detectable marker is linked are useful for diagnostic purposes. For example, a pharmaceutical composition containing a non-liposomal nanoparticle comprising a detectable label linked thereto is used to diagnose ischemia, e.g., before administration of the therapeutic particles. Any pharmaceutically acceptable detection agents that can be linked/conjugated to the nanoparticles are used. Suitable detectable labels are selected from the group consisting of a fluorescent organic dye, a radioactive molecule, and paramagnetic compound. A method of identifying an ischemic anatomical site in a subject is carried out by administering to a subject, e.g., a subject that is suffering from or suspected of suffering from ischemia, the detectably labeled nanoparticles described above and then imaging the subject or region of subject. The labeled nanoparticle preferentially localizes to an ischemic anatomical site compared to a non-ischemic site and the detection of the label indicates ischemia at that anatomical site.

Because nanoparticles localize to ischemic tissue via leaky vasculature (enhanced permeability and retention (EPR) effect), the size of the particles are tailored accordingly. For example, the nanoparticle comprises a diameter of less than 200 nm, e.g., a diameter of greater than 2 nm and less than 150 nm, e.g., a diameter of 5-100 nm, e.g., a diameter of 10-50 nm. Exemplary particles are less than 45, e.g., 40 nm, or less than 15 nm, e.g., 13 nm. The particles are comprised of any pharmaceutically acceptable material, e.g., silica or gold.

All polynucleotides and polypeptides of the invention are purified and/or isolated. Purified defines a degree of sterility that is safe for administration to a human subject, e.g., lacking infectious or toxic agents. Specifically, as used herein, an “isolated” or “purified” nucleic acid molecule, polynucleotide, polypeptide, or protein, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. Purity is measured by any appropriate standard method, for example, by column chromatography, polyacrylamide gel electrophoresis, or HPLC analysis. A purified or isolated polynucleotide (ribonucleic acid (RNA) or deoxyribonucleic acid (DNA)) is free of the genes or sequences that flank it in its naturally-occurring state.

By “substantially pure” is meant a nucleic acid, polypeptide, or other molecule that has been separated from the components that naturally accompany it. Typically, the polynucleotide, polypeptide, or other molecule is substantially pure when it is at least 60%, 70%, 80%, 90%, 95%, or even 99%, by weight, free from the proteins and naturally-occurring organic molecules with which it is naturally associated. For example, a substantially pure polypeptide may be obtained by extraction from a natural source, by expression of a recombinant nucleic acid in a cell that does not normally express that protein, or by chemical synthesis.

Small molecules include, but are not limited to, peptides, peptidomimetics (e.g., peptoids), amino acids, amino acid analogs, polynucleotides, polynucleotide analogs, nucleotides, nucleotide analogs, organic and inorganic compounds (including heterorganic and organomettallic compounds) having a molecular weight less than about 5,000 grams per mole, organic or inorganic compounds having a molecular weight less than about 2,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

By the terms “effective amount” and “therapeutically effective amount” of a formulation or formulation component is meant a sufficient amount of the formulation or component to provide the desired effect. By “an effective amount” is meant an amount of a compound, alone or in a combination, required to reduce or prevent ischemia in a mammal. Ultimately, the attending physician or veterinarian decides the appropriate amount and dosage regimen.

The terms “treating” and “treatment” as used herein refer to the administration of an agent or formulation to a clinically symptomatic individual afflicted with an adverse condition, disorder, or disease, so as to effect a reduction in severity and/or frequency of symptoms, eliminate the symptoms and/or their underlying cause, and/or facilitate improvement or remediation of damage. The terms “preventing” and “prevention” refer to the administration of an agent or composition to a clinically asymptomatic individual who is susceptible or predisposed to a particular adverse condition, disorder, or disease, and thus relates to the prevention of the occurrence of symptoms and/or their underlying cause.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All published foreign patents and patent applications cited herein are incorporated herein by reference. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### DESCRIPTION OF THE DRAWINGS

FIG. 1 is a series of photographs demonstrating ischemic-targeting of nanoparticles via the enhanced permeability and retention (EPR) effect in the murine ischemic hindlimb model. FIG. 1A is a photograph of control mouse hindlimbs. FIG. 1B is a photograph taken after ischemic surgery on the left hindlimb.

FIG. 2 is a series of a line graph, a photograph, and a bar graph demonstrating the temporal effect of targeting nanoparticles to ischemic tissues. FIG. 2A is a diagram of the timeline of ischemic surgery, nanoparticle injection, and imaging. FIG. 2B is a photograph of ischemic mouse hindlimbs at days 1, 3, 7, and 14 after ischemic surgery. FIG. 2C is a bar chart showing accumulation of nanoparticles in ischemic and healthy limbs.

FIG. 3 is a series of photographs showing that by inducing the temporary leakiness of a blood vessel, nanoparticles are delivered into tissues and/or organs. FIG. 3A is a photograph showing the results of active vascular endothelial growth factor (VEGF) delivery with alginate systems. FIG. 3B is a photograph showing the results of VEGF delivery via bolus injection. FIG. 3C is a photograph showing the results of VEGF delivery via intravenous injection.

FIG. 4 is a line graph showing the ischemia to non-ischemia blood flow ratio following VEGF delivery. Injection of gold nanoparticle-VEGF conjugates led to a significant recovery of perfusion.

FIG. 5 is a diagram showing ischemic tissue conditions in myocardial infarction, stroke, and peripheral vascular disease.

FIG. 6 is a diagram showing mechanisms of therapeutic angiogenesis with utilization of VEGF.

FIG. 7 is a diagram showing the enhanced permeability and retention (EPR) effect.

FIG. 8 is a diagram showing hypoxia-induced angiogenesis, and the similarity of tumor and ischemic tissue angiogenesis.

FIG. 9 is a diagram showing peripheral artery disease (PAD) and a model for PAD (femoral artery ligation). The lower right panel shows blood flow after nanoparticle-mediated therapeutic angiogenesis treatment.

FIG. 10 is a diagram showing the PEGylation of fluorescent silica nanoparticles.

FIG. 11 is a series of photographs of organs (from left to right: liver, spleen, lung, heart, kidney, and bladder) after administration of bare nanoparticles (bare NPs) and PEGylated nanoparticles (PEGylated NPs). PEGylated NPs showed lower signals in liver and spleen.

FIG. 12 is a series of photographs showing that PEGylated nanoparticles target ischemic limb tissue.

FIG. 13 is a series of photographs of mouse hindlimbs and line graphs summarizing ischemic/non-ischemic fluorescence and blood flow ratio over time following nanoparticle injection. Nanoparticles target more severe ischemic tissue.

FIG. 14 is a series of photomicrographs showing that severe ischemia induces higher expression of pVEGFR2, which results in blood vessel leakiness.

FIG. 15 is a diagram, line graph, and bar graph showing the effect of gold nanoparticle-mediated hVEGF165 delivery to ischemic tissue, as compared to VEGF bolus and bare nanoparticle delivery.

FIG. 16 is a line graph demonstrating the therapeutic effect of VEGF-conjugated gold nanoparticles in chronic ischemia, as compared to bare nanoparticle delivery.

### DETAILED DESCRIPTION

The invention provides a minimally-invasive method of intravenously injecting nanoparticles loaded with diagnostics and/or therapeutics into blood (systemic circulation) which subsequently accumulate in ischemic tissues for the purpose of identifying the location of ischemic sites in the body and for treating a variety of cardiovascular diseases. The accumulation of circulating nanoparticles into ischemic tissues is achieved by either passive targeting due to the enhanced permeability and retention (EPR) effect through the leaky vasculature induced by tissue ischemia, or by active targeting using specific ischemic tissue-targeting molecules coupled on nanoparticles. For example, local delivery of exogenous VEGF to a desired anatomical target site is useful as a method for directing nanoparticle accumulation to tissues of interest through the induction of temporal (or transient) leaky vasculature. The methods described herein are especially useful for developing diagnostics and therapeutic approaches for various ischemic diseases including cerebrovascular ischemia, renal ischemia, pulmonary ischemia, limb ischemia, ischemic cardiomyopathy, and myocardial ischemia, in which conventional invasive approaches can lead to adverse side effects.

### Ischemia

Cardiac ischemia may be asymptomatic or may cause chest pain, known as angina pectoris. It occurs when the heart muscle, or myocardium, receives insufficient blood flow. This condition frequently results from atherosclerosis, which is the long-term accumulation of cholesterol-rich plaques in the coronary arteries. Both large and small bowel can be affected by ischemia. Ischemia of the large intestine may result in an inflammatory process known as ischemic colitis. Ischemia of the small bowel is called mesenteric ischemia. Brain ischemia can be acute or chronic. Acute ischemic stroke is a neurologic emergency that may be reversible if treated rapidly. Chronic ischemia of the brain may result in a form of dementia called vascular dementia. Cutaneous ischemia occurs as a result of reduced blood flow to the skin layers may result in mottling or uneven, patchy discoloration of the skin. The methods are suitable for diagnosis, precise identification of ischemic anatomical locations or microenvironments, as well as treatment to improve/increase blood flow in such situations.

Hypoxia, the reduced oxygen availability of cells, is a potent inducer of upregulation of a variety of angiogenic factors in ischemic tissues through hypoxia-inducible factor 1 (HIF-1). Among various angiogenic factors, VEGF plays a key role in physiological and pathological angiogenesis. Cells activated by hypoxia produce VEGF that is able to attract inflammatory and endothelial cells, which initiate the neovascularization process to provide more nutrients and oxygen to hypoxic region. Leaky blood vessels are a characteristic of the initial stage of neovascularization. This key characteristic of a local environment of ischemic tissue (EPR effect) is used for nanoparticle-targeting to ischemic disease sites. The methods described herein are useful for noninvasive delivery of diagnostic molecules and therapeutic angiogenic molecules loaded in nanoparticles to ischemia tissue.

The targeted delivery of nanoparticles into ischemic tissue is achieved through conjugation of active targeting molecules on the nanoparticles. In response to ischemia and inflammatory mediators in several tissues, adhesion molecules, such as ICAM-1, P-selectin, E-selectin, and αᵥβ₃ integrin, are upregulated on endothelial cells. Such molecules (and their amino acid (aa) and nucleic acid (na) sequences) are well known in the art: ICAM-1 (GenBank Accession Number: (aa) CAA41977.1 (GI:825682), (na) NM_{—}000201.2 (GI:167466197), incorporated herein by reference), P-selectin (GenBank Accession Number: (aa) AAQ67703.1 (GI:34420913), (na) NM_{—}003005.3 (GI:157419153), incorporated herein by reference), E-selectin (GenBank Accession Number: (aa) AAQ67702.1 (GI:34420911), (na) NM_{—}000450.2 (GI:187960041), incorporated herein by reference), and αᵥβ₃ integrin (GenBank Accession Number: (aa) 1JV2_A (GI:16975253; chain A), (na) L28832.1 (GI:454817; integrin beta 3), incorporated herein by reference). Thus in addition of passive targeting via EPR effect that mediates localization of the nanoparticles to an ischemic site, conjugation of an antibody or small peptide that targets/binds to the adhesion molecules to the surface of nanoparticles, is a means of active targeting of nanoparticle to ischemic tissue.

### Nanoparticles for Targeting of Ischemic Tissue

The methods described herein are useful as a noninvasive nanoparticle-targeting strategy to diagnose and treat areas of ischemia in the body. A variety of materials are useful for making nanoparticles, e.g., silica, polymer, metal, metal oxide, liposome, and quantum dots, and etc. Nanoparticles less than 200 nm in diameter are preferable for targeting of ischemia. The nanoparticles used for diagnostic purposes are coupled with various molecules including fluorescent organic dye, radioactive molecules, and paramagnetic compounds for imaging. Therapeutic nanoparticles are linked to various growth factors such as VEGF, PDGF, and bFGF to promote therapeutic angiogenesis. Such molecules (and aa and na sequences) are well known in the art. Exemplary factors include vascular endothelial growth factor (e.g., VEGFA; GenBank Accession Number: (aa) AAA35789.1 (GI:181971), (na) NM_{—}001171630.1 (GI:284172472), incorporated herein by reference), basic fibroblast growth factor (bFGF; GenBank Accession Number: (aa) AAB21432.2 (GI:8250666), (na) A32848.1 (GI:23957592), incorporated herein by reference), platelet derived growth factor (PDGF; GenBank Accession Number: (aa) AAA60552.1 (GI:338209), (na) NM_{—}033023.4 (GI:197333759), incorporated herein by reference), placental growth factor (PLGF; GenBank Accession Number: (aa) AAH07789.1 (GI:14043631), (na) NM_{—}002632.4 (GI:56676307), incorporated herein by reference), Angiopoietin (e.g., Ang-1; GenBank Accession Number: (aa) AAI52420.1 (GI:156230950), (na) NM_{—}001146.3 (GI:21328452), incorporated herein by reference), stromal-derived factor (e.g., SDF-2; GenBank Accession Number: (aa) AAP35355.1 (GI:30582257), (na) NM_{—}006923.2 (GI:14141194), incorporated herein by reference), granulocyte-macrophage colony stimulating factor (GM-CSF; GenBank Accession Number: (aa) AAA52578.1 (GI:183364), (na) M11220.1 (GI:183363), incorporated herein by reference, and granulocyte colony stimulating factor (G-CSF; GenBank Accession Number: (aa) CAA27290.1 (GI:732764), (na) X03438.1 (GI:31689), incorporated herein by reference).

The particles are administered to the body using known methods such as intravenous, intraperitonal, intramuscular, or intrathecal infusion or injection or by direct administration to a desired tissue or organ. Any systemic method of administration is suitable for the methods described herein. For example, particles are administered locally (e.g., at or 0.1, 1, 2, 5, or 10 cm from the affected ischemic site). For example, particles are administered locally, regionally (e.g., >10 cm, such as 15, 20, 50, 75, 100 cm from the ischemic site), or systemically (e.g., anywhere in the body relative to the location of the ischemic site. Systemic administration is typically intravenous infusion or injection. In another example to achieve directed targeting of nanoparticles to a tissue of interest, injectable polymeric gels incorporated with growth factors, especially VEGF, are injected locally to a target anatomical site to induce temporal vessel leakiness. The nanoparticles are then injected into the body intravenously for targeted delivery to ischemia or interested tissue with temporally/transiently induced leaky vasculature.

### EXAMPLE 1

### In Vivo Targeting of Fluorescent Silica Nanoparticles to Ischemic Tissue via Enhanced Permeability and Retention (EPR) Effect

Polyethylene glycol (PEG) was conjugated to nanoparticles and in vivo localization was monitored. PEGylation of nanoparticles was achieved by using different PEG molecules depending on the nanoparticles. For example, PEG-silane was conjugated on the surface of silica nanoparticles (SiNPs) through silane chemistry, and PEG-SH was used for Au nanoparticles through covalent bonding between Au and thiol group. Other suitable methods of interaction include electrostatic interactions. PEGylation methods are well known in the art.

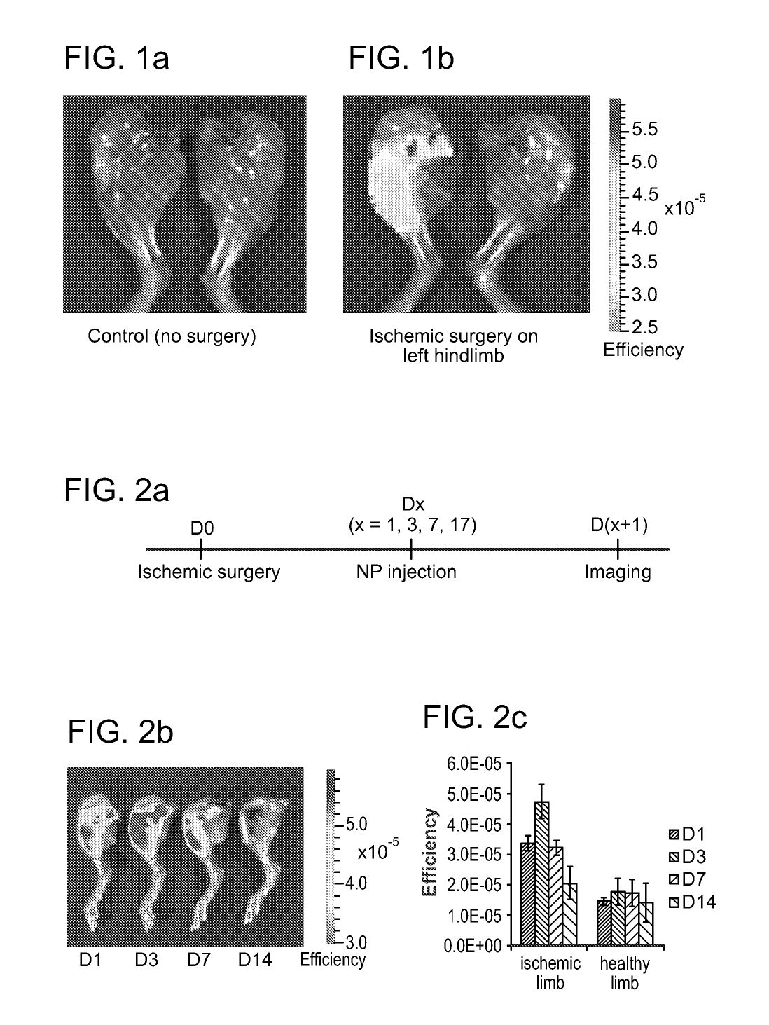

To verify ischemia-targeting of nanoparticles via the EPR effect, PEGylated fluorescent silica nanoparticles doped with Cy5.5 dye with size of 40 nm were injected intravenously into control mouse (FIG. 1A) and murine ischemic hindlimb model (FIG. 1B) 1 day after ischemic surgery and the limbs were imaged ex vivo under Xenogen bioimaging systems. The fluorescent images of the ischemic and normal hindlimb tissues indicated that the significant targeting of nanoparticles to the ischemic muscle occurred following injection of PEGylated nanoparticles. There was a strong fluorescence in the ischemic muscle with injection of fluorescent silica nanoparticles, but not in the normal muscle, indicating that the method of intravenously injected nanoparticles resulted in preferential delivery to ischemic muscle rather than normal muscle. This result indicates that enhanced secretion of multiple angiogenic factors including VEGF mediated by hypoxia makes the surrounding blood vessels become leaky, thus allowing the circulating nanoparticles to escape from the blood stream and accumulate in the nearby tissue region.

### EXAMPLE 2

### Temporal Effect of Targeting Nanoparticles to Ischemic Tissue

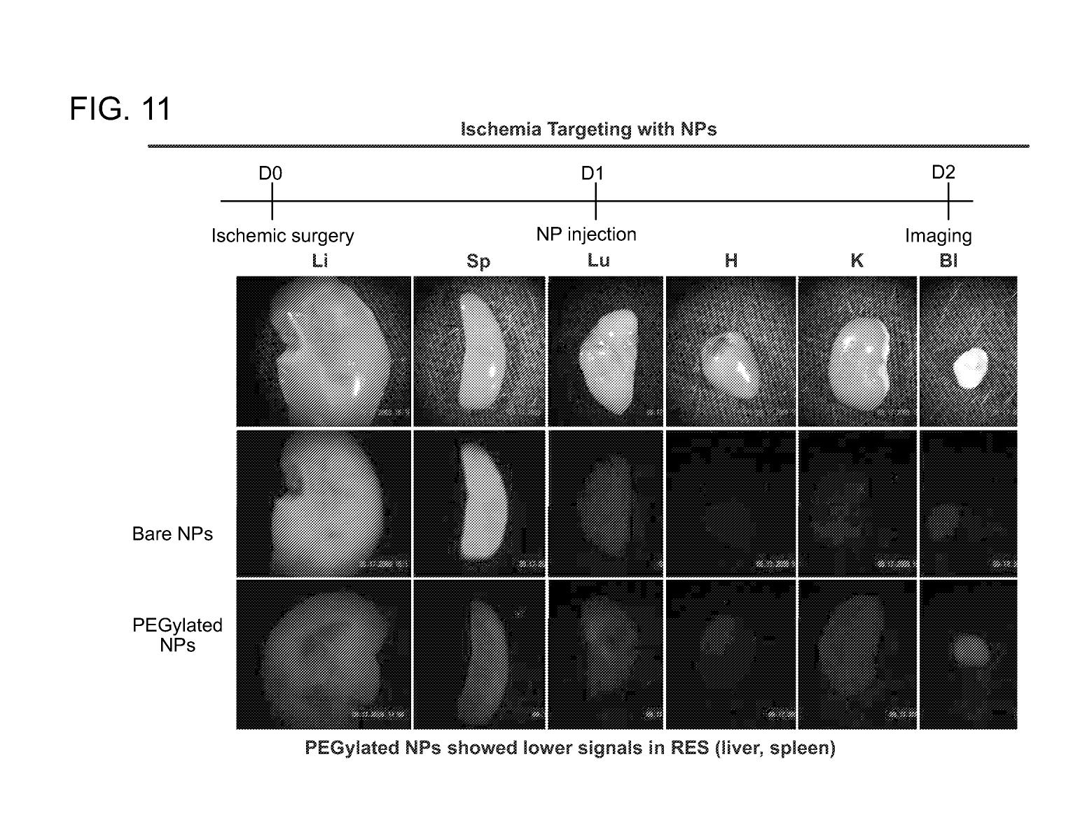

Biodistribution of the nanoparticles following administration was studied. To evaluate the biodistribution of PEGylated nanopartices, PEGylated R-SiNPs were injected intravenously to murine ischemic hindlimb model one day after ischemic surgery. Bare R-SiNPs (unPEGylated) were used as a negative control of PEGylated R-SiNPs. FIG. 11 shows the biodistribution of the nanoparticles based on the fluorescence intensity of the accumulated nanoparticles in major organs including liver, spleen, lung, heart, kidney, and bladder. Fluorescence images for the bare R-SiNPs showed much higher fluorescences in the reticuloendothelial system (RES) such as liver and spleen as compared with the PEGylated R-SiNPs, indicating PEGylation of the silica nanoparticles led to a higher stability and a longer circulation time in the blood, by avoiding being trapped in the RES. The fluorescence signal in the bladder for the PEGylated nanoparticles compared with insignificant fluorescence for the bare nanoparticles indicate that the PEGylated nanoparticles are excreted through urine after circulation in the blood. These results also indicate that the colloidal stability of NPs through surface modification, such as PEGylation, is important for ischemia targeting.

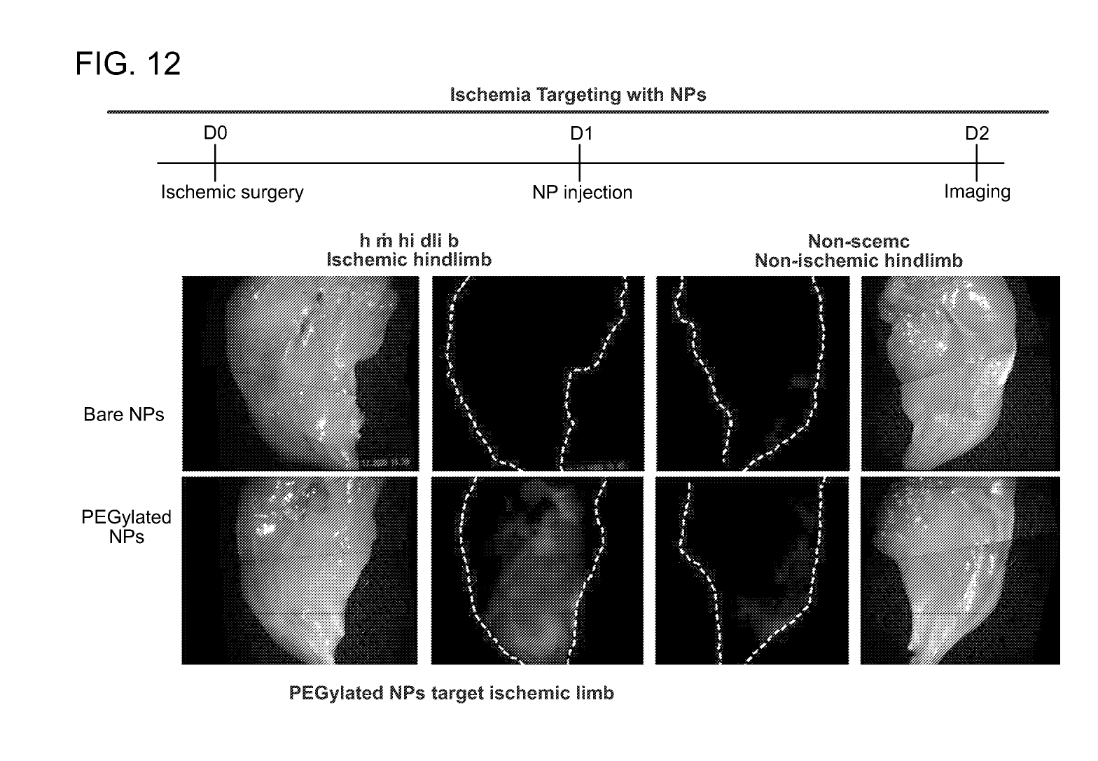

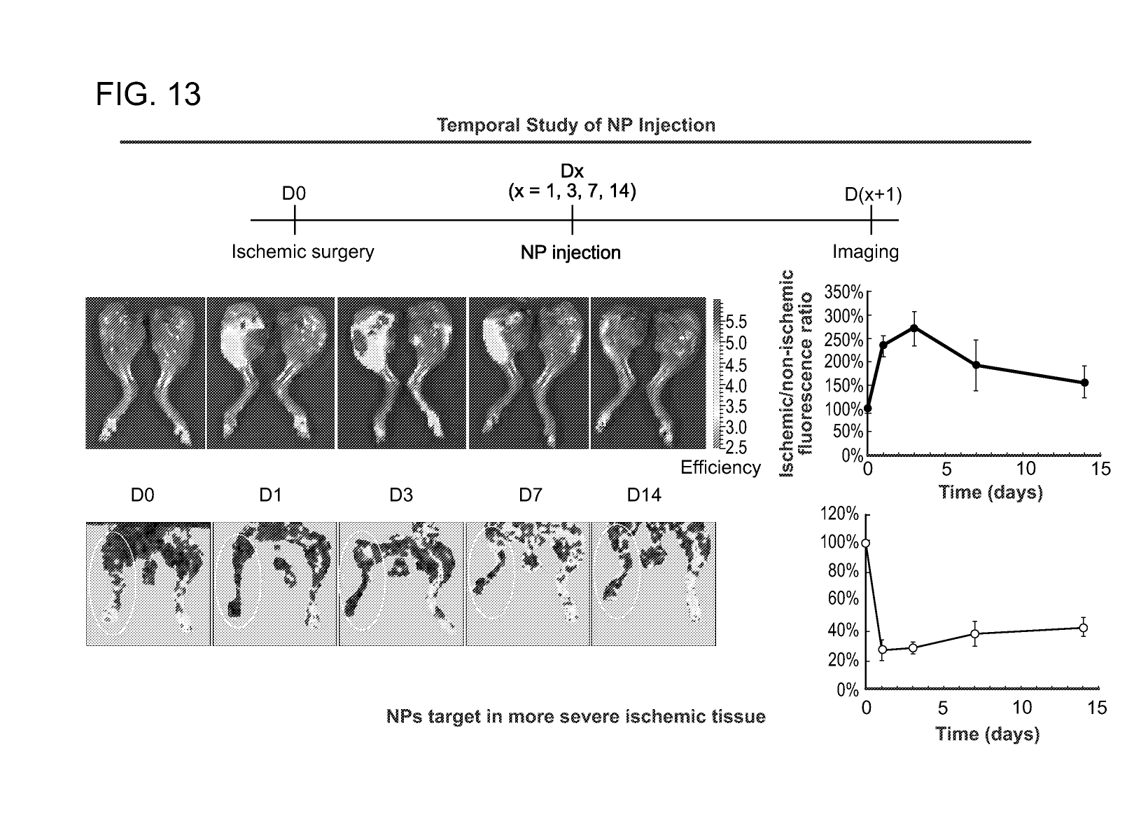

The temporal effect of targeting of nanoparticles to ischemic tissues was further investigated as shown in FIG. 2. PEGylated silica nanoparticles were injected intravenously through tail vein at day 1, 3, 7, or 17 (n=3) after ischemic surgery (day 0), and the ischemic and normal hindlimb were imaged 24 h after nanoparticle-injection (FIG. 2A). The representative fluorescent images of ischemic hindlimb showed mostly a higher accumulation of nanoparticles into ischemic hindlimbs compared with normal hindlimbs at day 1, 3, and 7 (FIG. 2B and 2C). At day 14, the nanoparticle accumulation was decreased close to the fluorescence level of normal limb (FIG. 2B and 2C). These data indicate that the injection of nanoparticle before day 7 after ischemic surgery provides a significant accumulation of nanoparticles into ischemic muscle. Moreover, the results presented in FIGS. 12-13 demonstrate that PEGylated nanoparticles target ischemic limbs and more severe ischemic tissue.

### EXAMPLE 3

### Targeted Delivery of Therapeutic Nanoparticles is Mediated by Blood Vessel Leakiness

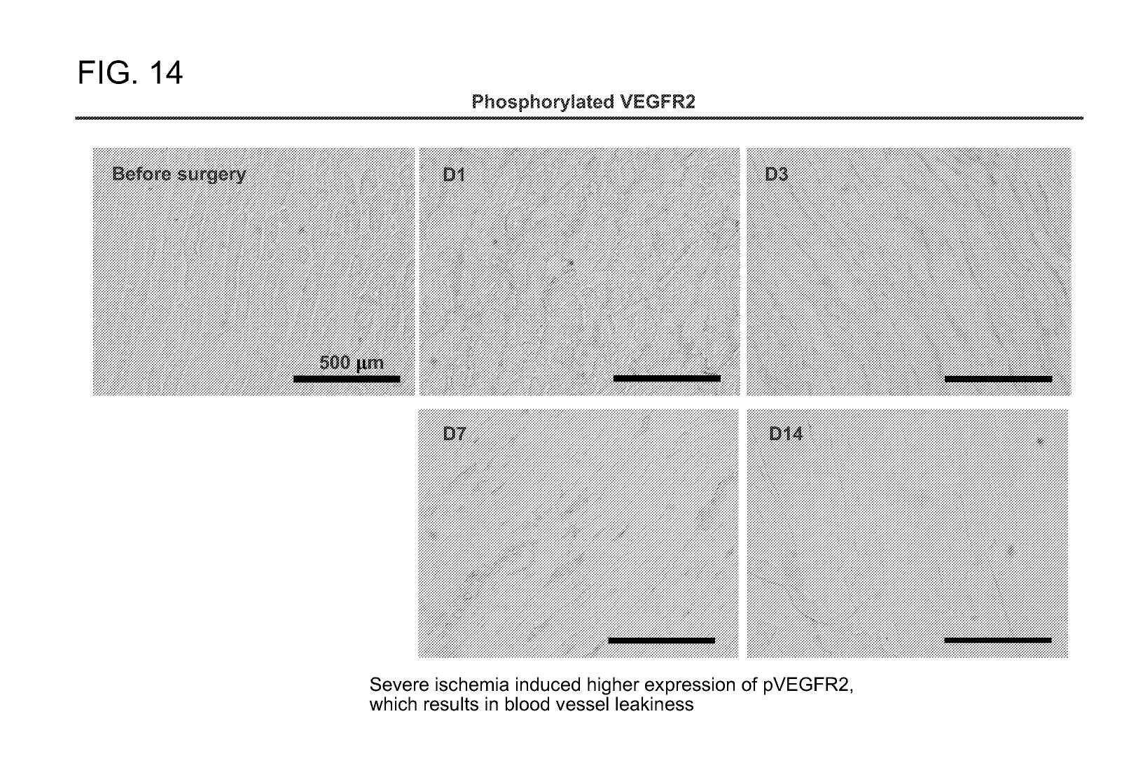

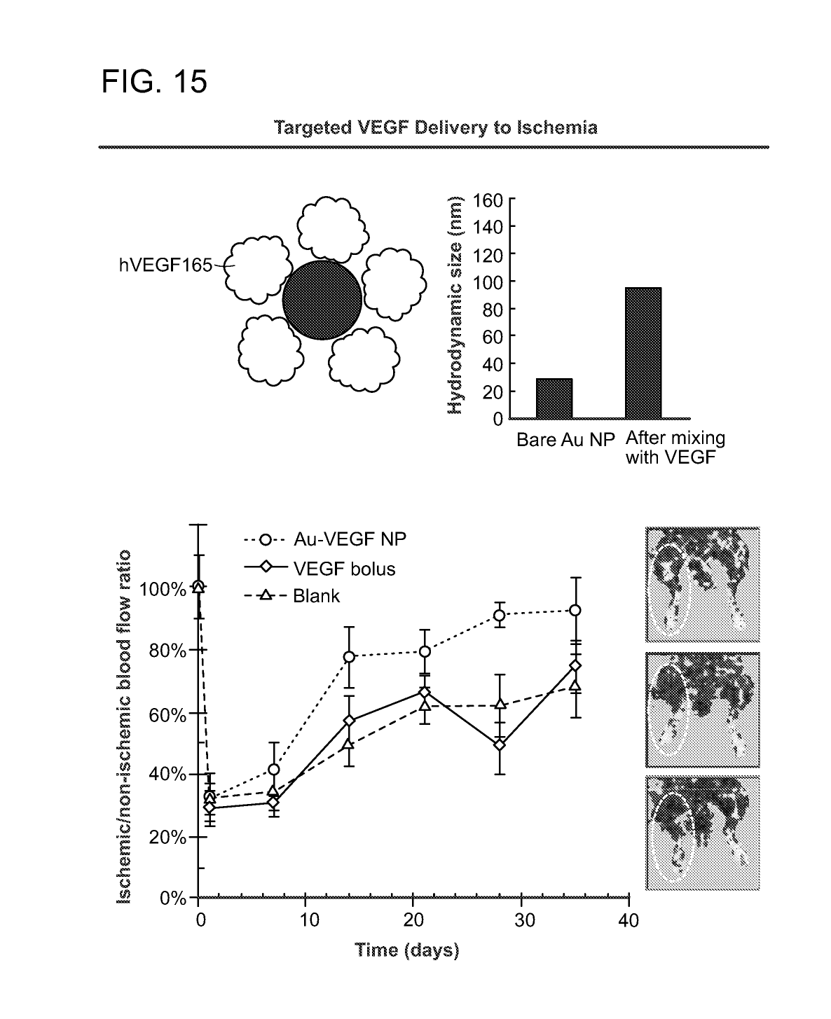

The nanoparticle-targeting strategy to the ischemic muscle via the EPR effect opens up the accessibility of nanoparticles to various muscle diseases. The VEGFR2 can be activated to phosphorylated form upon exposure with VEGF, which shows a connection between VEGF signaling and leakiness of blood vessels. The expression of pVEGFR2 was checked with immunostaining. Higher expression in D1 and D3 than D14 was observed (FIG. 14), which supports the higher NP accumulations in early time point after ischemic surgery.

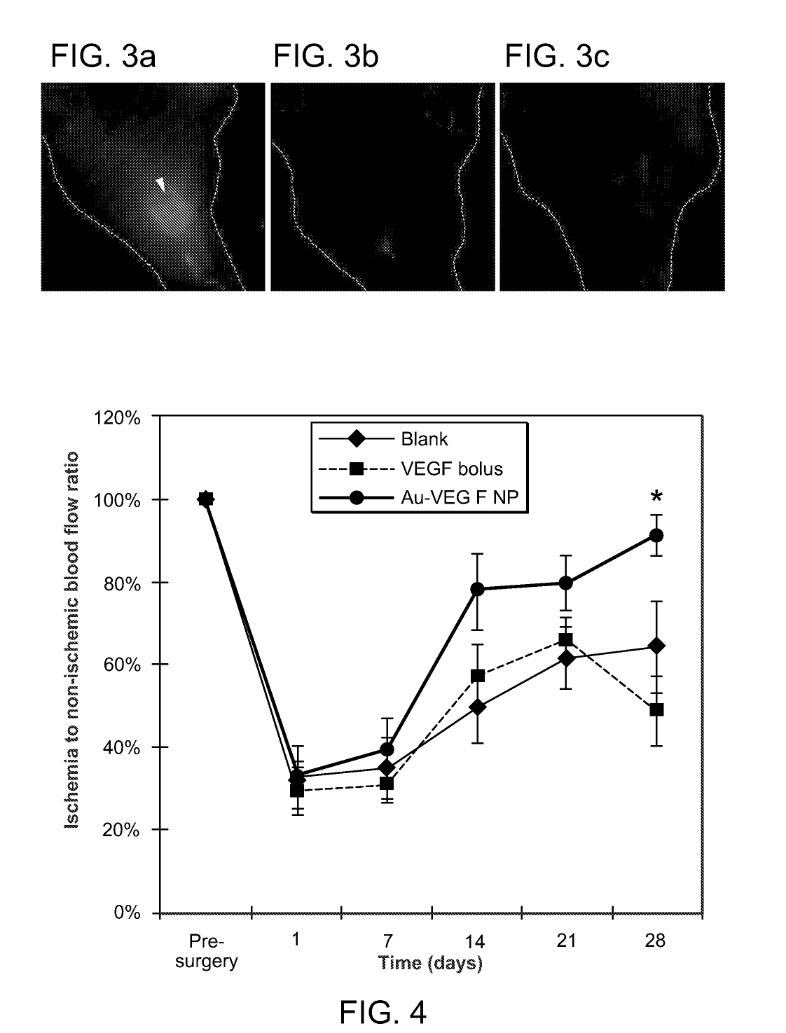

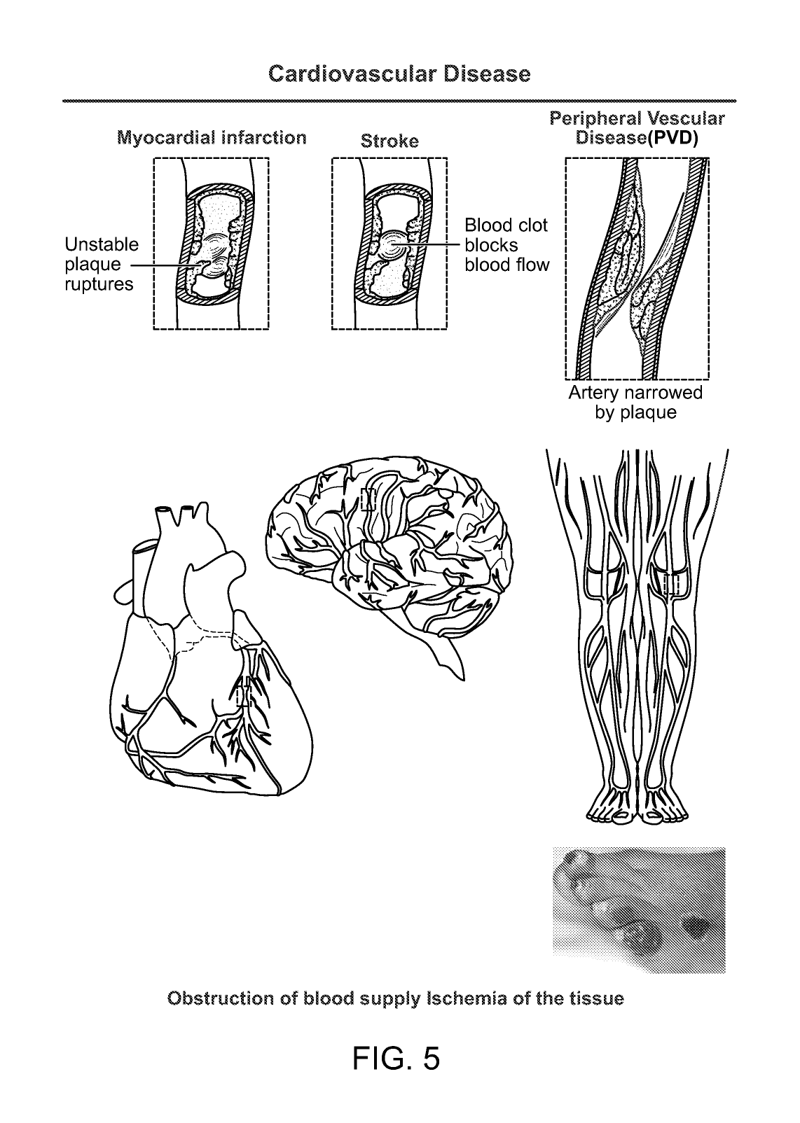

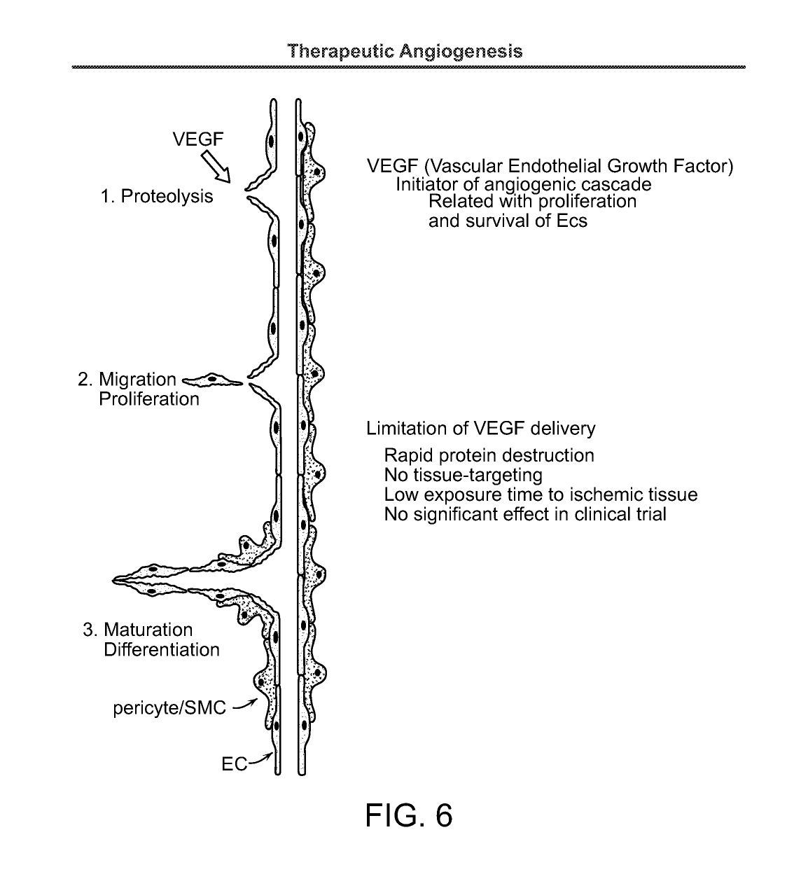

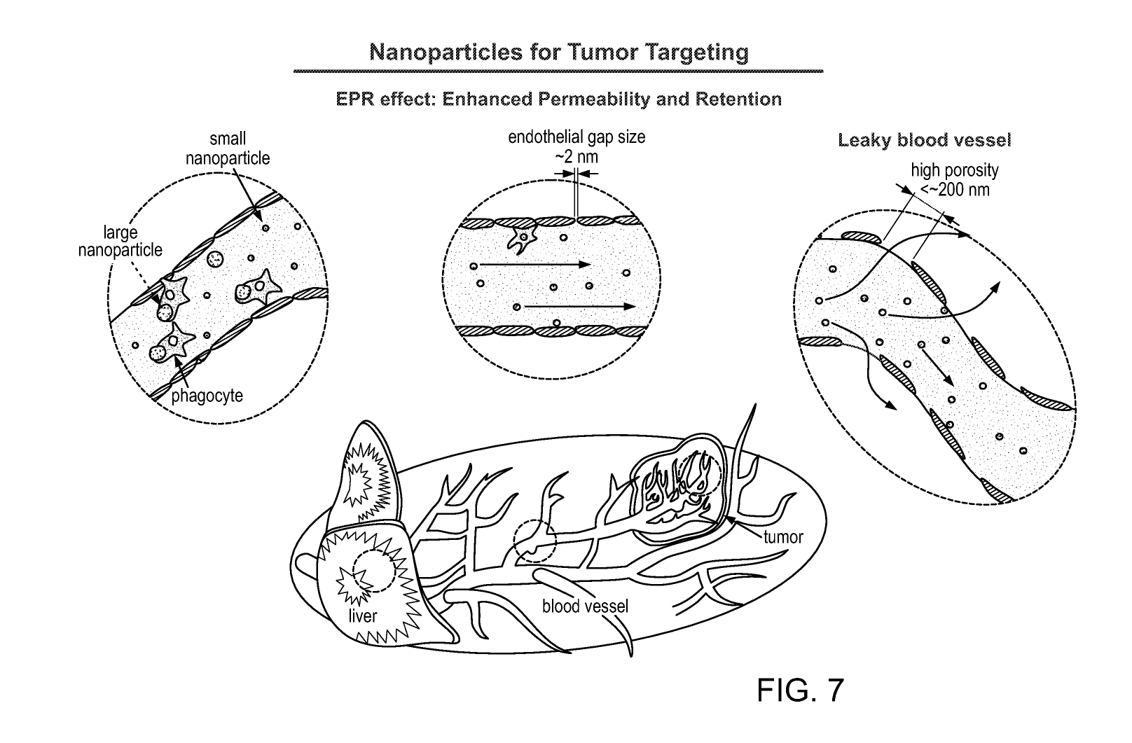

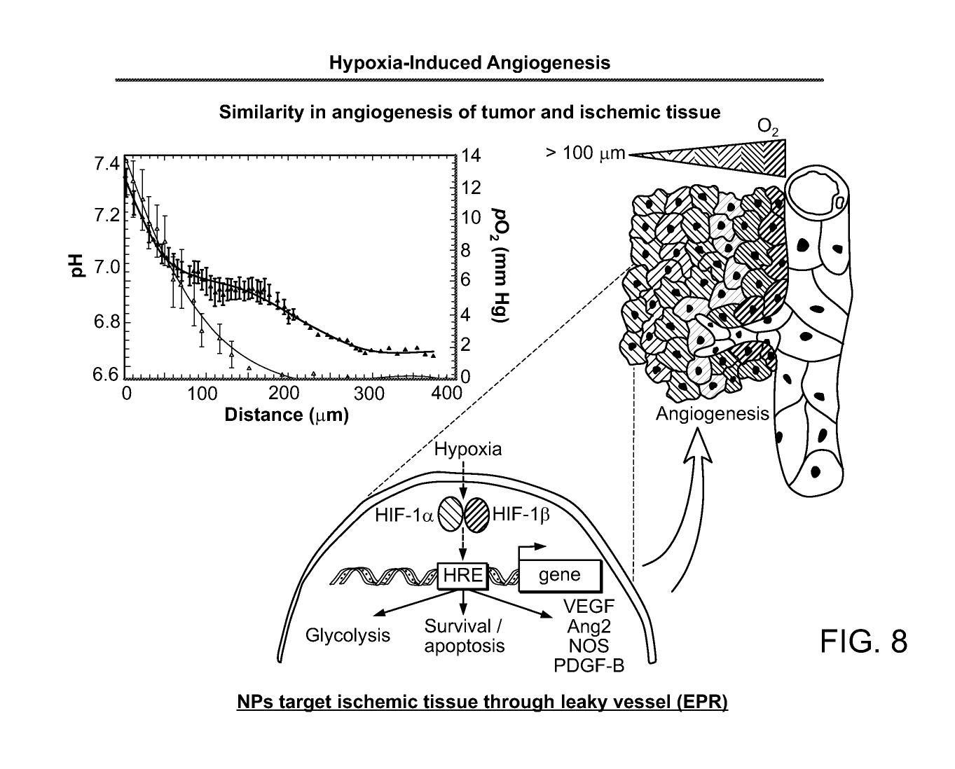

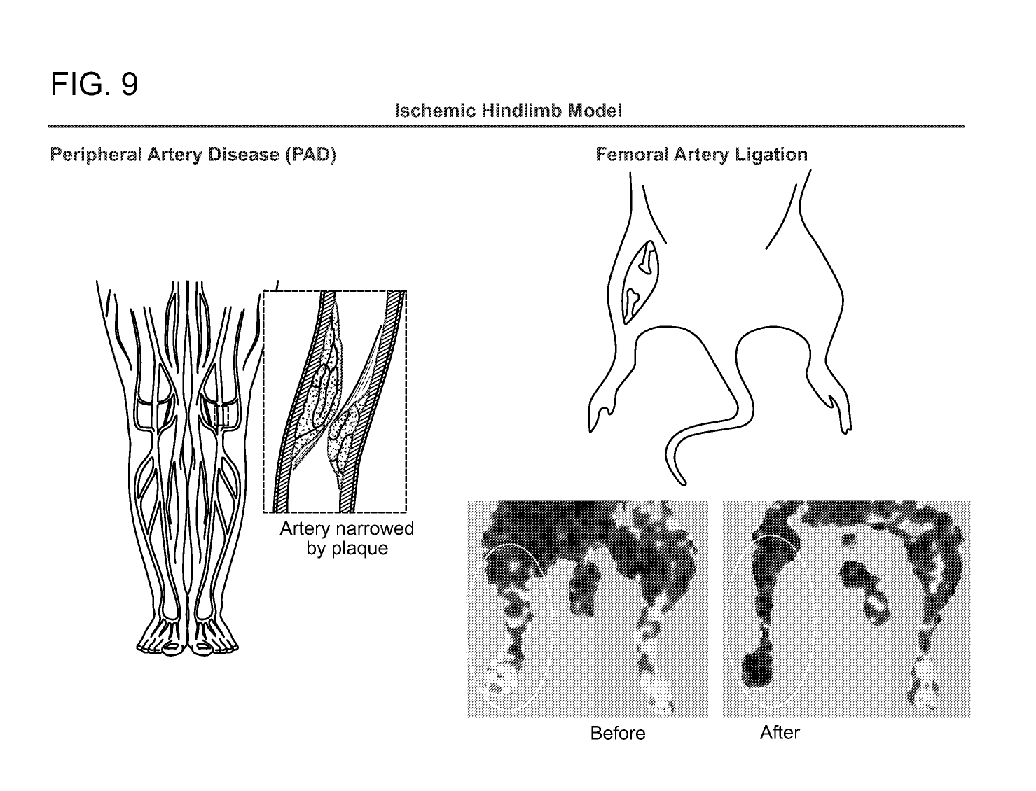

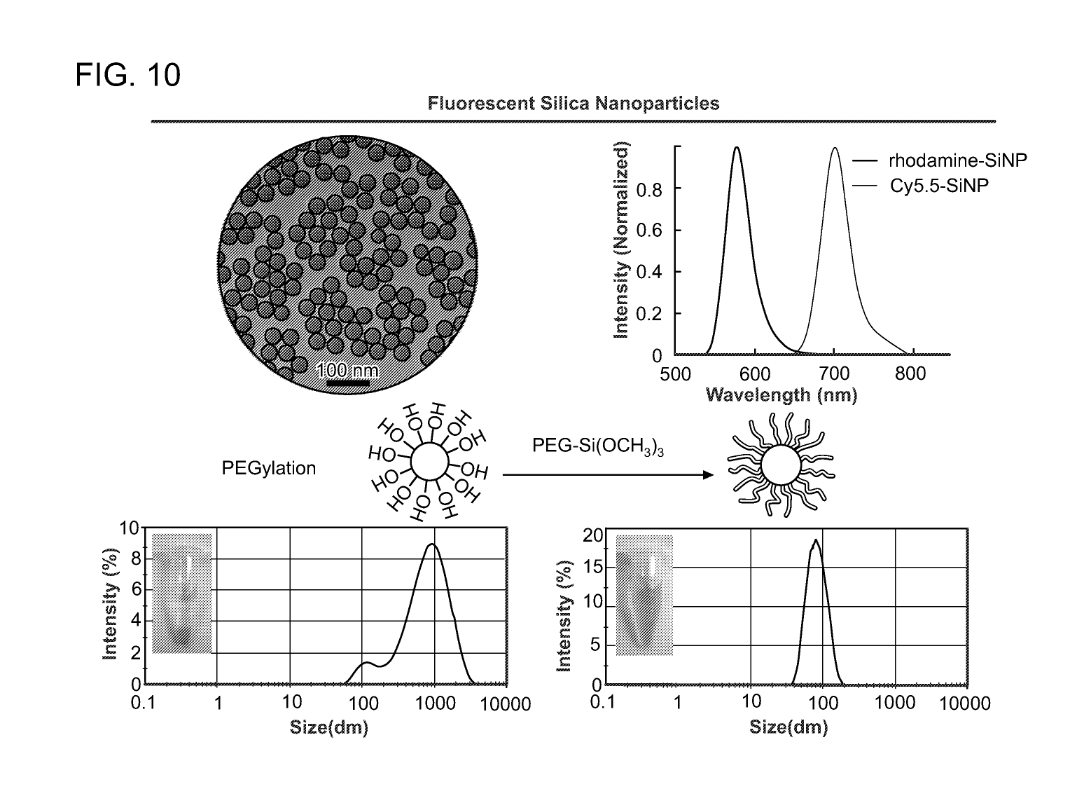

The ability to induce a microenvironment of leaky blood vessels temporarily at target tissues allows the targeted delivery of therapeutic nanoparticles with payload via the EPR effect. To create such a transient microenvironment of EPR, VEGF was used as a triggering agent of temporal leakiness of blood vessel. VEGF were delivered into the normal hindlimb muscle of the healthy mouse with injectable VEGF-alginate system (FIG. 3A), bolus injection (FIG. 3C), or intravenous injection (FIG. 3C). The fluorescent silica nanoparticles were injected into the blood stream at day 1 and the hindlimbs were imaged at day 2 post surgery. The fluorescent image showed that fluorescent nanoparticles were selectively delivered into the muscle through the active VEGF delivery with alginate systems (FIG. 3A). These data indicate that nanoparticles were targeted into a muscle tissue of interest through artificial triggering or induction of vessel leakiness. In contrast, there was no significant accumulation of nanoparticles in the muscle from bolus injection (FIG. 3B) and intravenous injection (FIG. 3C) of VEGF. These results indicate that triggering temporary leakiness of blood vessels in a target tissue or organ leads to delivery/localization of the nanoparticles with payload to that location.

### EXAMPLE 4

### Delivery of Therapeutic Payload using Nanoparticles to Ischemic Tissue

To evaluate the delivery of therapeutic payload using nanoparticles to ischemic tissue, gold nanoparticles were tested as a model of nanoparticle-carrier system. Conjugation of VEGF on gold (Au) NPs was carried out as follows. The disulfide groups in VEGF were utilized for the conjugation of VEGF on the surface Au nanoparticles through covalent bonding between Au atom and thiol groups. The size of conjugated nanoparticles in dynamic light scattering was ˜100 nm, which is in good size regime for extravasation through leaky blood vessels.

In further experiments, 13 nm gold nanoparticles conjugated with 3 μg of VEGF were injected into murine hindlimb ischemic model (n=8) and the blood flow of the ischemic hindlimb as a ratio of the non-ischemic counter-lateral limb was checked with Laser Doppler Perfusion Imaging (LDPI) to investigate the functional recovery of ischemic hindlimbs. Gold nanoparticle-VEGF was injected at day 1 post-induction of ischemia (acute ischemic model). Blank (n=8) and intravenous injection of 3 μg of VEGF (n=4) were studied as control. Compared to the i.v. injection of VEGF and blank, injection of gold nanoparticle-VEGF conjugates led to a significant recovery of perfusion at 4 weeks after injection (FIG. 4). These data indicate that noninvasive delivery of proangiogeneic factors using nanoparticles as carriers successfully led to localization of the proangiogenic payload at the target site and led to physiological improvement (e.g., increased blood flow) at that site.

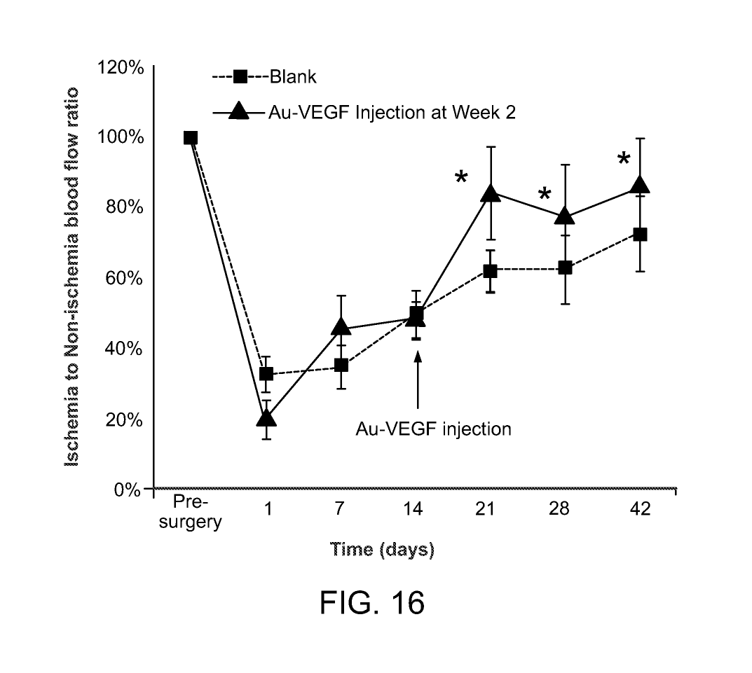

Many human patients with peripheral arterial disease (PAD) exhibit chronic ischemic status. To examine the therapeutic effect of VEGF-conjugated gold nanoparticles in chronic ischemia, the nanoparticles were injected 2 weeks post-induction of ischemia (chronic ischemic model). Murine hindlimbs were injected with gold nanoparticles conjugated with 3 μg of VEGF (n=9). The blood flow ratio of the ischemic hindlimb compared to the non-ischemic hindlimb was examined utilizing Laser Doppler Perfusion Imaging (LDPI) to investigate the functional recovery of ischemic hindlimbs. Blank (n=8) with saline injection were studied as control. Compared to blank control limbs, injection of gold nanoparticle-VEGF conjugates at week 2 post-induction of ischemia led to a significant recovery of perfusion beginning 1 week after injection (FIG. 16). These results demonstrate that the nanoparticles loaded with VEGF targeted the ischemic tissue even 2 weeks after induction of ischemia, which indicates the utility and efficacy of the claimed methods in treatment of chronic or disseminated ischemia.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

1. A pharmaceutical composition comprising a non-liposomal nanoparticle comprising an angiogenesis-promoting factor linked thereto.

2. The composition of claim 1, wherein said factor is a growth factor or cytokine.

3. The composition of claim 1, wherein said factor is selected from the group consisting of vascular endothelial growth factor (VEGF), basic fibroblast growth factor (bFGF), platelet derived growth factor (PDGF), placental growth factor (PLGF), Angiopoietin, stromal-derived factor (SDF), granulocyte-macrophage colony stimulating factor (GM-CSF), and granulocyte colony stimulating factor (G-CSF).

4. The composition of claim 1, wherein said factor is VEGF.

5. The composition of claim 1, wherein said nanoparticle further comprises a directed targeting composition that binds to or associates with ICAM-1, P-selectin, E-selectin, or αᵥβ₃ integrin.

6. The composition of claim 5, wherein said directed targeting composition is an antibody or fragment thereof.

7. A method for preferentially promoting angiogenesis at an ischemic anatomical site compared to a non-ischemic site, comprising administering to a subject identified as suffering from or suspected of suffering from ischemic and administering to said subject the composition of claim 1, wherein said nanoparticle preferentially localizes to said ischemic anatomical site compared to said non-ischemic site.

8. A method for promoting angiogenesis at a target anatomical site in a subject, comprising locally administering to said target site an EPR-inducing agent and subsequently administering to said subject the composition of claim 1.

9. A pharmaceutical composition comprising a non-liposomal nanoparticle comprising a detectable label linked thereto.

10. The composition of claim 9, wherein said detectable label is selected from the group consisting of a fluorescent organic dye, a radioactive molecule, and paramagnetic compound.

11. A method of identifying an ischemic anatomical site in a subject, comprising administering to said subject the composition of claim 9 and imaging said subject, wherein said nanoparticle preferentially localizes to said ischemic anatomical site compared to a non-ischemic site and wherein the detection of said label indicates ischemia at said ischemic anatomical site.

12. The composition of claim 1, wherein said nanoparticle comprises a diameter of less than 200 nm.

13. The composition of claim 1, wherein in said nanoparticle comprises a diameter of greater than 2 nm and less than 150 nm.

14. The composition of claim 1, wherein said nanoparticle comprises a diameter of 5-100 nm.

15. The composition of claim 1, wherein in said nanoparticle comprises a diameter of 10-50 nm.
